(19) United States
(12) Patent Application Publication
(10) Pub. No.: US 2013/0046168 A1
(43) Pub. Date: Feb. 21, 2013

(54) METHOD AND SYSTEM OF CHARACTERIZATION OF CAROTID PLAQUE

(71) Applicant: Lei Sui, Medina, WA (US)

(72) Inventor: Lei Sui, Medina, WA (US)

(21) Appl. No.: 13/211,487

(22) Filed: Aug. 17, 2011

(51) Int. Cl. A61B 8/00; A61B 5/055; A61B 5/00

(52) U.S. Cl. 600/411; 600/407; 600/437

(57) ABSTRACT

A system and method of obtaining and analyzing ultrasound images of a patient provides for the identification of specific tissue types in using the image data. A feature vector set of sub-regions of the region of interest is obtained, dimensionally reduced and evaluated using a heuristic to identify the tissue type. Where the tissue type is suitable for image standardization, the overall gray scale of the image is adjusted with respect to a predetermined gray scale for the identified tissue type. The image may be segmented and plaque regions identified and characterized. The characterized plaque and other parameters such as percent stenosis may be used to determine a risk score for the patient.

This invention was partially supported by the National Institutes of Health under Contract No. HL103387. The Government has certain rights in the invention.

### TECHNICAL FIELD

The present application may relate to imaging, detection, characterization, monitoring, and risk stratification in medical imaging.

### BACKGROUND

Carotid atherosclerosis is a pathological build-up of fatty materials on carotid artery wall. The build-up usually has a fibrous cap and necrotic core (NC). Carotid atherosclerosis is a slow initially asymptomatic prognosis which eventually becomes symptomatic and may lead to cardiovascular or neurovascular events, depending on the characteristics of the plaque. Research has shown that the morphological, compositional, mechanical, electromagnetic properties and surrounding hemodynamics may have significant diagnostic effect.

Conventional treatment of carotid atherosclerosis includes medication, stenting, and endarterectomy. The selection criteria for treatment are cardio- or neurovascular symptoms and the degree of stenosis. Stenosis is an abnormal narrowing of the vascular channel. Unfortunately, these criteria do not appear to be good indicators of vulnerable plaque that may cause stroke.

Medical ultrasound imaging has been the screening tool of choice so as to identify the degree of stenosis. Typically, a Duplex ultrasound, spectral Doppler plus 2D B or BC mode ultrasound images, estimates the degree of stenosis from the blood speed measured by the Doppler gate in the carotid lumen, and the location or size of the plaque from the B or BC mode images. Experienced sonographers may also qualitatively estimate the degree of stiffness of the plaque based on the subjective acoustic image appearance. Patients are then assigned to different treatment paths according to their Duplex ultrasound screening results. Despite the improvement of medical ultrasound imaging, the technique does not yet provide a reliable estimate on the plaque vulnerability.

There are several factors that prevent medical ultrasound from reliably estimate the vulnerability. Consistent imaging settings across a population of patients is yet to be achieved. The arbitrary location of the 2D imaging plane and subjective setting of imaging parameters such as gain, for example, make it difficult to identify and extract features characteristic of vulnerable plaque. Moreover current approaches do not provide a quantitative measurement of plaque attributes. Echolucency (transparency of the echo), smoothness and vessel wall stiffness have been associated with vulnerability. But, there is no standard quantitative measure of these three descriptors, or a specific observational means of quantifying the criteria. Moreover, there is no consistent set of criteria of vulnerability even if such measurements could be made.

At present the plaque may often characterized by using Magnetic Resonance Imaging (MRI). US PgPub 20100106022 “CAROTID PLAQUE IDENTIFICATION METHOD” describes an algorithm for analyzing the brightness of an ultrasound plaque image and the thickness of fibrous cap of the plaque to classify the plaque into high or low risk. Though the mechanism of plaque vulnerability is not fully understood, recent histological studies are suggestive that the vulnerability is associated with the following arterial characteristics: a) large homogeneous lipid-rich necrotic core (LR/NC); b) thin fibrous cap; c) active inflammation with hemorrhage or neovasculature; d) severe stenosis; d) endothelial denudation with superficial platelet aggregation and fibrin deposition. Noninvasive techniques for accurately identifying vulnerable plaques (also called “high-risk” plaques) would aid in stroke-risk stratification and cost-effective therapeutic intervention.

### BRIEF SUMMARY

Herein, an apparatus and method for charactering carotid plaque as a function of its morphological, mechanical, electromagnetic and hemodynamic properties using ultrasound or other non-invasive imaging modalities and a structured interactive strategy for using the measured characteristics is described. In particular, the method may, for example, begin with a step of characterization of the plaque using an imaging modality of low cost and easy access such as ultrasound (US), and continue, if needed to a step of further other modalities such as MRI or CT (computerized tomography), or to a step of making a diagnosis and selecting a treatment path. For example, ultrasound (US) may be used to screen-out low-risk patients and refer other patients for more detailed but more expensive analysis, such as MRI or CT (computed tomography). The results from ultrasound may be interactively combined with the imaging results from MRI or CT to form a more complete evaluation of the patient.

Disclosed herein are methods and systems for standardizing aspects of imaging in ultrasound imaging and automating analysis of carotid plaque shown in such images, with or without human intervention in the analysis process.

In an aspect, a system and method is provided for standardizing the brightness of observed carotid lumen and surrounding tissue across an ensemble of patients during or subsequent to ultrasound image acquisition. The system and method also makes the observed speckle pattern consistent across an ensemble of patients for ultrasound imaging.

The speckle characteristic in an ultrasound image may be subject to texture analysis and may be related to specific tissue types. Tissue type identification is a basis for an image standardization technique that mitigates the variation of image characteristics of existing US techniques, and generalizes ultrasound imaging so as to be subject to computer aided segmentation and analysis.

In another aspect, a method is provided for automated identification of plaque in an image of a person. The existence of plaque may be characterized, for example, as 1) protrusion of vessel wall into carotid lumen which narrows the lumen; or 2) thickness of intima-media layer of the vessel wall bigger than 0.5 mm With the acquisition of data from one or more imaging modality, which may be associated in space or time, the identification of lumen, vessel wall and plaque can be automated. The composition of the identified plaque may also be characterized.

For example, the vessel wall boundary may be estimated from the blood flow profile or the tissue displacement velocity pattern during a cardiac cycle. This vessel wall boundary estimate may serve as the initial lumen boundary for further processes. A plurality of image types may be aligned in space or time, and the images may be obtained by a plurality of imaging modalities. Physical device location methods, timing using absolute time, cardiac, and relative time may be used to select, fuse and analyze the image data. Cross-correlation of signals from multiple images may be used. Where the term “image” is used, a person of skill in the art would understand that this is also intended to refer to the data set from which an image, a trace, or other representation of the data set may be produced. Different types of ultrasound image data herein means, for example, B-mode, tissue velocity or flow velocity images or volumes, and their radio frequency (RF) or I and Q representations from the RF acoustic data, with or without envelope detection, contrast enhancement or scan conversion spectral Doppler or M-mode traces.

Automation may include signal processing and pattern recognition techniques. Brightness quantification may be adaptive based on the local region appearance, or may be analytic (e.g. texture analysis) with the imaging settings computed at the time of data acquisition, or subsequently. Texture analysis may include, for example, a plurality of texture computations at multiple resolutions or distances from Haralick texture features, gray level difference features, run-length features and Laws texture features. The overall texture features can be brightness/gain independent or dependent with or without dimension reduction. Dimension reduction preserves the most significant information at the minimal sufficient dimensions. The multi-level pattern recognition and classification of the plaque from the above features may be rule based or statistical-model based. The process scale size of the characterization may be, for example, multi-scaled from pixel, to small regions, or the whole plaque structure. The automation process can be edited by human intervention to correct algorithmic errors or improve the accuracy.

In an example, the apparatus and method may result in the output acquired data, processed data and analysis results, or a combination thereof, by a display device, electronic media or hard copy, in their original format or pseudo-color-coded format. The data may be transferred by electronic storage media, data network or hard copy for comparison to other test results. The processed data may include the intermediate quantification, classification and risk score in the form of text, graphs, 2D (two-dimensional) images, 3D (three dimensional) volume at a particular time and location, or in a series of time and location images to show retrogression or progression of the syndrome.

In another aspect, the ultrasound data may be combined with data from other imaging modalities for integrated diagnosis and follow up. In yet another aspect, a method is provided for automated identification and optimization of the carotid lumen boundary in 3D with ultrasound imaging. A B-mode 2D image and a color or B blood flow mode 2D image may be acquired in which images are geometrically and temporally registered. A series of such B mode slices and blood flow slices may be used to form 3D volumes. The blood flow profile determined from the flow component may provide an initial location of lumen boundary, which may be further defined by edge detection or region segmentation in the B mode component. The observed lumen boundary can be further refined by manually editing the image.

In a further aspect, a method is described for determining blood flow volume using ultrasound images through a cardiac cycle. The blood flow volume may be overlaid with a corresponding B volume. Multiple images covering a cardiac cycle may be acquired at locations along, for example, the carotid artery. The acquisition may be gated, with or without a timing device or positioning controls. The acquisition volume may be slowly scanned by moving the acoustic transceiver of the ultrasound imaging device along the carotid artery such that the image data covers a predefined number of cardiac cycles of the targeted volume. Then, the image data may be sorted according to their temporal position with respect to the cardiac cycle which may be determined either by a timing device, such as an ECG, or by signal processing. This process results in a series of carotid volumes spaced throughout the cardiac cycle.

In yet another aspect, a method is described for integrating data and information from different modalities in diagnostic decision making and planning of patient care. For example, the blood flow data from ultrasound images may clarify the interpretation of shadows in MRI image, which may be from flow motion, plaque or calcification.

An ultrasound diagnostic system is described, including an ultrasound device producing image data of a patient; a computer in communication with the ultrasound device, the computer configured to process the image data to obtain a plurality of feature vectors characterizing a region of the image. The feature vectors are dimensionally reduced and used to identify a specific tissue type based on a heuristic.

A method of analyzing ultrasound data is described, including the steps of obtaining an ultrasound image of a region of interest for a patient; determining a set of feature vectors for a sub-region of the region of interest; and dimensionally reducing the feature vector set and identifying a tissue type of the sub-region using a heuristic. Where the identified tissue type is suitable for image standardization, image gray scale value are standardized with respect to a predetermined mean gray scale for the identified tissue type, by adjusting the overall gray scale of the image.

In another aspect, computer program product, stored in a non-transient computer-readable medium, includes instructions interpretable by a computer to cause the computer to: accept image data image of a region of interest for a patient; determine a set of feature vectors for sub-regions of the region of interest; dimensionally reduce the feature vector set and identify a tissue type of the sub-regions using a heuristic; wherein when the identified tissue type is suitable for image standardization, standardize the mean gray scale of the sub-region with respect to a predetermined mean gray scale for the identified tissue type by adjusting the overall gray scale of the image.

### BRIEF DESCRIPTION OF THE DRAWINGS

FIG. 1 illustrates a B-scan ultrasound image having texture feature values characteristic of tissue, plaque and noise, that may be used for algorithm training;

FIG. 2 is a graph of the reduced feature set vectors of the selected regions of FIG. 1;

FIG. 3 is a sonogram of a breast where the gray scale of a region has been standardized;

FIG. 4 is an ultrasound image of a carotid artery, where a plaque region has been segmented from the surrounding tissues and regions of the plaque having different echogenetic properties are further differentiated;

FIG. 5 is the same ultrasound image as FIG. 4, where a plaque region having calcification has been segmented and a region of shadowing may be identified below the plaque;

FIG. 6 is another ultrasound image of a carotid artery where the top version shows two identified regions of interest, and the bottom shows one of the top regions of interest in greater detail where the fibrous cap has been delineated;

FIG. 7 is a simplified system block diagram for an ultrasound system configured to perform the disclosed methods (a network interface to either the ultrasound device or the processor is not shown);

FIG. 8 shows a flow diagram for a method of acquiring, standardizing and characterizing an ultrasound image;

FIG. 9 shows a method for acquiring and assembling 3 D images over a cardiac cycle;

FIG. 10 shows a block diagram of a method for characterizing patient risk; and

FIG. 11 shows a method of determining a course of treatment, or the need for further diagnosis based on the patient risk as determined by ultrasound image analysis.

### DETAILED DESCRIPTION

Exemplary embodiments may be better understood with reference to the drawings. In the interest of clarity, not all the routine features of the implementations described herein are described. It will of course be appreciated that in the development of any such actual implementation, numerous implementation-specific decisions must be made to achieve a developers' specific goals, such as compliance with system, business or regulatory constraints, and that these goals will vary from one implementation to another.

The combination of hardware and software to accomplish the tasks described herein is termed a system. Where otherwise not specifically defined, acronyms are given their ordinary meaning in the art.

The instructions for implementing processes or methods of the system may be provided on computer-readable storage media or memories, such as a cache, buffer, RAM, removable media, hard drive or other computer readable storage media. Computer readable storage media include various types of volatile and nonvolatile storage media, where the storage of data is non-transient. The functions, acts or tasks illustrated in the figures or described herein are executed in response to one or more sets of instructions stored in or on computer readable storage media, or distributed thereon. The functions, acts or tasks are independent of the particular type of instruction set, storage media, processor or processing strategy and may be performed by software, hardware, integrated circuits, firmware, microcode and the like, operating alone or in combination. Likewise, processing strategies may include multiprocessing, multitasking, parallel processing, grid processing, and the like.

In an embodiment, the instructions may be stored on a removable media device for reading by local or remote systems. In other embodiments, the instructions may be stored in a remote location for transfer through a computer network, a local or wide area network, or over telephone lines. In yet other embodiments, the instructions are stored within a given computer or system.

The instructions may be a computer program product, stored or distributed on computer readable storage media, containing some or all of the instructions to be executed on a computer to perform all or a portion of the method or the operation of the system.

Herein a processor or a computer is meant to include, as needed, a central processor unit (CPU), working memory, appropriate storage media for data and software, network interfaces, including wireless interfaces, Internet and LAN, input and output data terminals, displays, and the like, as is known in the art. The processor may be a single device or distributed amongst the tangible elements of the system.

Where the term “data network”, “web” or “Internet” is used, the intent is to describe an internetworking environment, including both local and wide area networks, where defined transmission protocols are used to facilitate communications between diverse, possibly geographically dispersed, entities, including cluster computers on a campus, or a wide area network, or the like. An example of such an environment is the world-wide-web (WWW) and the use of the TCP/IP data packet protocol, and the use of Ethernet or other known or later developed hardware and software protocols for some of the data paths.

Communications between the devices, systems and applications, and an interface to a data network, may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions.

Where the term “wireless” is used, it should be understood to encompass a transmitting and receiving apparatus, a transceiving apparatus, or the like, including any antennas, and electronic circuits for modulating or demodulating information onto an electrical signal, which may subsequently be radiated or received. The term wireless, when describing an apparatus, does not encompass an electromagnetic signal in its free-space manifestation. A wireless apparatus may include both ends of a communications circuit or only a first end of a circuit where another end of the circuit is a wireless apparatus interoperable with the wireless apparatus at the first end of the circuit. Many connections between equipment may be either wired or wireless, depending on the specific design approach chosen.

In an aspect, the system and method makes use of the differing texture features associated with differing tissue types as measured by ultrasound imaging of a person or an animal.

Before discussing the various texture features of an ultrasound image it may be helpful to clarify the terms segmentation, classification and feature measures, as used herein. Segmentation is used to refer to the process of dividing an image up into substantially homogeneous regions according to some homogeneity criteria. It is therefore also concerned with establishing the boundaries between these regions without regard to the type or class of the regions. Such boundaries and the identification of tissue types, and the like, may be on the basis of heuristics. Herein, the term “heuristic” is intended to mean a selection criteria based on either experimental data or analysis of a structure or image that may be used to effectively distinguish between two alternate hypotheses. This may be a parameter such as a size, a range of sizes, relative sizes, a grey scale threshold, or the like, and ultimately related to for example, tissue type.

Classification refers to the process of grouping domains of image features into classes, where each resulting class contains samples meeting some similarity criterion (a heuristic). If the classes have not been defined a priori, the task may be referred to as unsupervised classification. Alternatively, if the classes have already been defined (normally through the use of training sets of sample textures which may be grouped on the basis of similarity, histological examination, or previously accepted work) then the process may be referred to as supervised classification. Herein, the classification is generally of the supervised type, unless specifically noted. However both methods may be used.

An image having regions of differing textures serving as features may be segmented using the features prior to, or subsequent to classification. That is, for example, the boundary between differing tissue-type regions may be established based on a heuristic and regions comprising the same tissue type separately identified from regions having other tissue types. This may be presented to a user by a pseudo-color display, by showing outline boundaries, by shading, or by other visual or electronic means. When the classification of images regions by tissue type is performed on a pixel-by-pixel or similar small-scale basis within a single image then, as a by-product, effective segmentation of the image also occurs as a result of the classification.

To perform the segmentation or the classification, some homogeneity or similarity criteria may be defined for each tissue type of sub-type. These criteria are normally specified in terms of a set of feature measures, which each of provide a quantitative measure of a certain characteristic texture features of the tissue. These feature measures are may be referred to here as texture measures features, or textures. Where the feature measures are analyzed for purposes of segmentation or classification purposes the feature measures may be referred to as feature vectors.

Ultrasound images may exhibit a variety of textures. Such textures may be expressed as feature vectors and characterized as representing particular tissue types, at least heuristically. One method of texture analysis is a so-called Haralick feature analysis. This is a gray-scale co-occurrence matrix (GLCM). Such GLCM analysis may be used to quantify the number of occurrences, at various distances and angles, of pixel intensity values with respect to each other. Using such analysis techniques, image features as angular second moment, contrast, sum average, sum variance, inverse difference moment, sum of squares (variance), entropy, sum entropy, difference entropy, difference variance, correlation, and maximum correlation coefficient may be calculated. These may be the raw feature vectors obtained from analysis of the pixels of an image.

Selection among extracted image features encompasses tradeoffs between desired properties. For example, a higher order of moment invariant provides more sensitivity but may make the features more susceptible to noise. Feature vector space reduction may be performed to select the most distinctive features. Feature reduction may be divided into categories, for example: feature selection, in which features carrying the most information are picked out through some selection scheme, or feature recombination, in which some features are combined (e.g., with different weights) into a new (independent) feature.

The dimensionality of the feature vectors obtained may be reduced by techniques such as principal component analysis (PCA), non-linear iterative partial least squares (NIPALS), stepwise discriminant analysis (SDA) or other similar methods in order to plot the data in a two or three dimensional form and to visualize data clusters representing different tissue or structure types.

The feature vectors may be clustered by unsupervised machine learning methods such as K-Mean clustering, Ward's hierarchical clustering, Kohonen's self-organizing maps, or similar methods. The feature vectors may be also classified by supervised learning methods such as linear or quadratic discriminant analysis (LDA, QDA), neural networks (NNs), or support vector machines (SVM).

Features for the classification of echolucency and heterogeneity of the plaque may be selected from mean, standard deviation, variation index, entropy and skewness of the pixel/voxel gray scales in the plaque. Other measures may be used as well.

$$\begin{matrix}Mean & GSM = {\sum }_{i} p , i \\ Standard deviation ( Stdv ) & Stdv = \sqrt{{\sum }_{i} {{p}_{i} ( i - GSM )}^{2}} \\ Variation index ( VI ) & VI = \frac{Stdv}{GSM} \\ Entropy ( E ) & E = {\sum }_{i} {p}_{i} ln {p}_{i} \\ Skewness ( S ) & S = \frac{1}{{Stdv}^{3}} {\sum }_{i} {{p}_{i} ( i - GSM )}^{3}\end{matrix}$$

where pᵢ is the probability of gray scale i in the plaque region.

The expected characteristics of some of the tissues being imaged by ultrasound are likely to be as shown in Table I. This preliminary characterization is based on an assessment of previously reported literature.

TABLE I

| Plaque Core Material | Mean | Stdv | VI | E | S |
|---|---|---|---|---|---|
| Lipid no hemorrhage | Low | Low | Mid | Low | Low |
| Lipid with hemorrhage | Mid | Mid | High | Mid | Big |
| Fibrous | High | Mid | Low | High | Low |

The artery may be identified as the space between the lumen-intima interface (lumen boundary) and the media-adventitia interface (wall boundary). Interior to the lumen boundary blood flow may be observed, depending on the type of US image being processed.

As observed with ultrasound, lipids and blood are low echogenic materials. Carotid artery plaques with rich lipid and hemorrhage are more echolucent than others with calcification and fibrous tissues. Conventional US imaging may not appropriately differentiate lipid from hemorrhage in the plaque in the visual analysis of an US image; however, an accurate assessment of echogenicity would have useful clinical implications, as several published studies have shown that echolucent and heterogeneous carotid plaques are associated with increased risk for cerebrovascular events.

Previously, echogenicity was usually evaluated subjectively. Subjective evaluation uses the observed intensity of local blood vessel and lumen in the image as reference. The intensity of the segmented plaque and its surrounding tissues were categorized into hypoechoic, isoechoic or hyperechoic according to observer's visual perception. Such subjective evaluation tends to have a large variability. It is also quite dependent on the settings of the US device and operator technique.

Objective evaluation has been used to calculate the mean or median gray scale (GSM) of the segmented plaque and its surrounding tissues after gray scale standardization. A threshold was applied to classify the plaque as a whole entity into echolucent or echogenic. Objective evaluation by this method marginally reduces the variability, but may not be sufficient repeatable for diagnostic purposes. The 2D imaging plane on a 3D object is difficult to reproduce exactly as the exact orientation of the sensor head may be poorly controlled. Moreover, the presence of shadows due to calcification, for example, may interfere with operator judgment in subjective evaluations.

Using feature analysis, the carotid artery and the surrounding tissue may be differentiated, so as to identify the outer boundary of the vessel. Similarly, the lumen boundary may be identified using feature vector analysis, Doppler (color) images, or the like.

Speckle is a characteristic image phenomenon in laser, radar, or ultrasound images. Its effect is to impart a granular aspect to the image. Speckle is understood to be an image artifact caused by interference between coherent waves that, backscattered by natural particles or structures within an imaging volume arising from small scale structure, arrive in phase or out of phase at the sensor for a given voxel (three dimensional pixel volume). Speckle tends to hamper the perception and extraction of fine details in the image by an operator. Consequently, in most instances, the objective of image data processing is to suppress the speckle. However, if the speckle pattern features of an area of the ultrasound image can be related to a particular tissue type then not only may the tissue types be segmented, but the value of the gray scale may be standardized so as to improve the repeatability of US images and to automatically classify the types of tissues in the image. Usually, the only use to which image speckle is put is to study the dynamics of displacement, stress and strain via speckle tracking.

One may use the technique of feature analysis to characterize small areas of the ultrasound image such as a pixel, a speckle, or a group of pixels in terms of texture, for example, speckle related features, and gray scale difference features, so as to differentiate between different types of tissue. This may be accomplished by comparing the characterizable features in a voxel with those of surrounding voxels and to collapse the feature vector space so as to focus on the most characteristic image features, using feature analysis techniques. A training set of data may be obtained, and the salient feature sets associated with tissue types identified by histological techniques. Alternatively, for example, a plurality of images where previous work has identified such tissue differentiation based on morphological criteria may also be used.

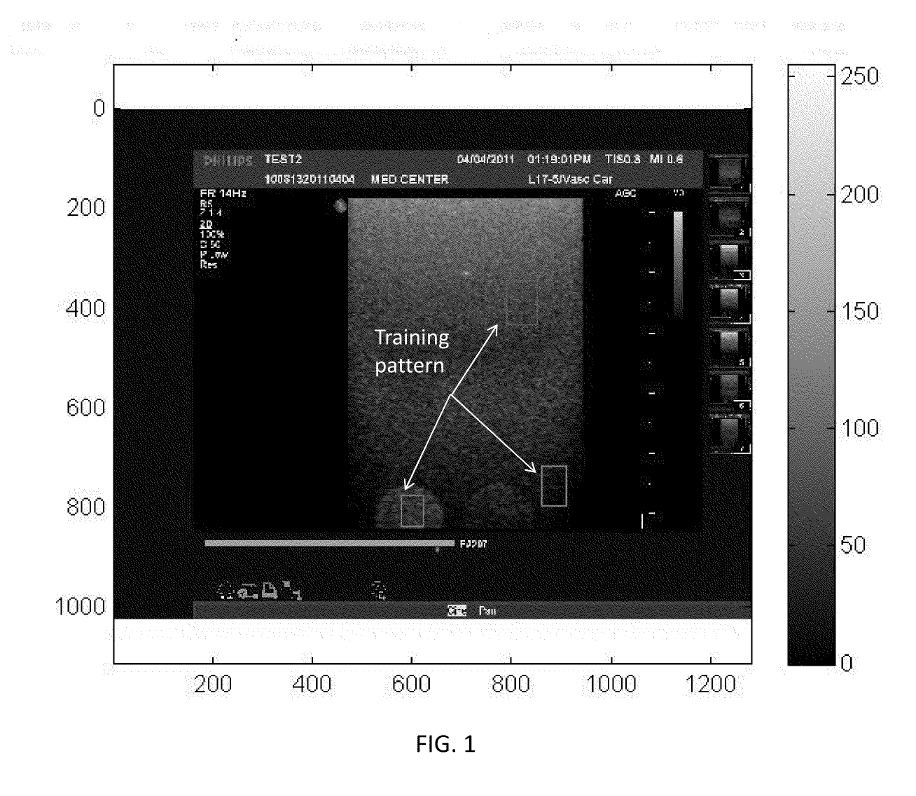

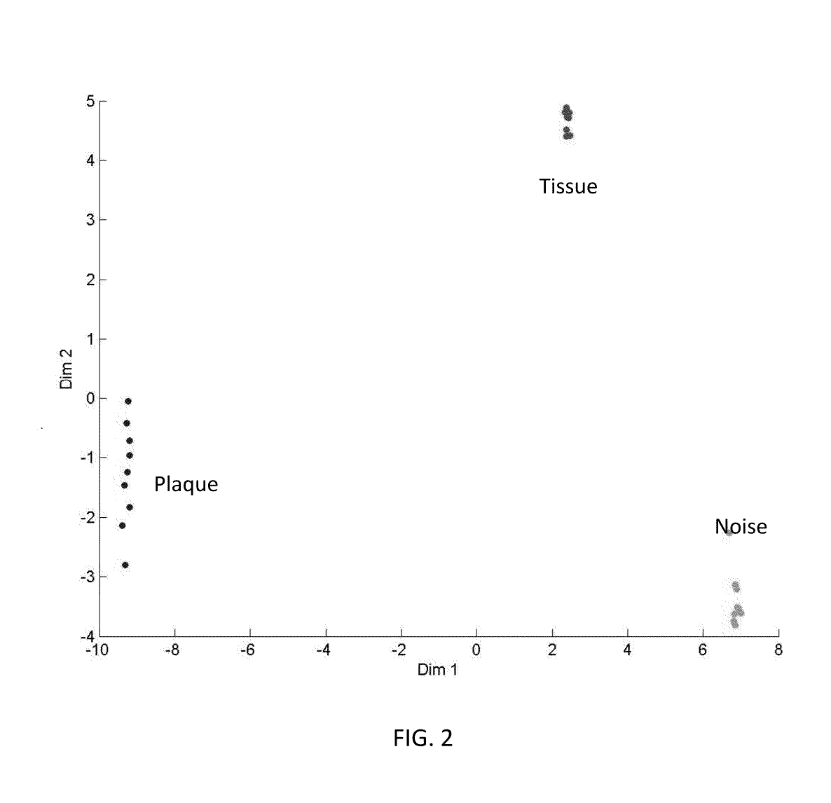

FIG. 1 is a B-scan sonogram showing a simulated training pattern. Three speckle patterns are shown and are and may be considered as representative of tissue, plaque and noise. The boxed areas correspond to the areas where feature analysis may be performed. After dimensional reduction, the feature vectors are plotted in FIG. 2

Each of the selected areas in FIG. 1 may be analyzed so as to determine a set of representative feature vectors for a contiguous region of voxels. The feature vectors are seen to cluster in differing regions of the feature space. Where the characteristics of the grouping of feature sets are sufficiently different, a region around each feature set in feature space may be established as being representative of a tissue type. After training using a number of representative images, the composite feature set clusters may serve to define the heuristic for identifying the tissue types in the ultrasound image.

Having been categorized by tissue type, a mean and covariance scattering value may be associated with the tissue type, typically associated with the density of the tissue. The general body tissue surrounding the vessel may be considered to be the most stable estimate of a mean scattering value, as there is likely to a reasonably large relatively undifferentiated tissue volume whose characteristics are not strongly dependent on the illumination angle. So, after identifying the body tissue region in the image by classification, segmentation, or the equivalent thereof, the gain or sensitivity of the ultrasound device may be automatically or manually adjusted to provide for an image where the mean scattering value of body tissue corresponds to a particular gray scale value. Other higher order characteristics of the tissue types may be used. While the largest dynamic range may be obtained when this gain adjustment is made when the image is being obtained, it will be appreciated that this technique may be used on previously obtained images.

The mean gray scale value, for example, corresponding body tissue may vary with depth into the body, primarily due to the attenuation of the ultrasound signal. Other variations may be due to shadowing by calcification or variations in the angle of the sensor or the coupling efficiency. Using the tissue type identification, the mean gray scale, or other characteristic of the image pixels may be corrected for depth if desired. Such normalization while imaging may be performed once for a group of images, or for each image independently. This process enables a standardized sensitivity to be used, independent of operator preference, room lighting (for image interpretation), coupling of the sensor to the patient, and the like. Similar normalization may be performed on previously obtained image data that is retrieved from a data base or other storage medium.

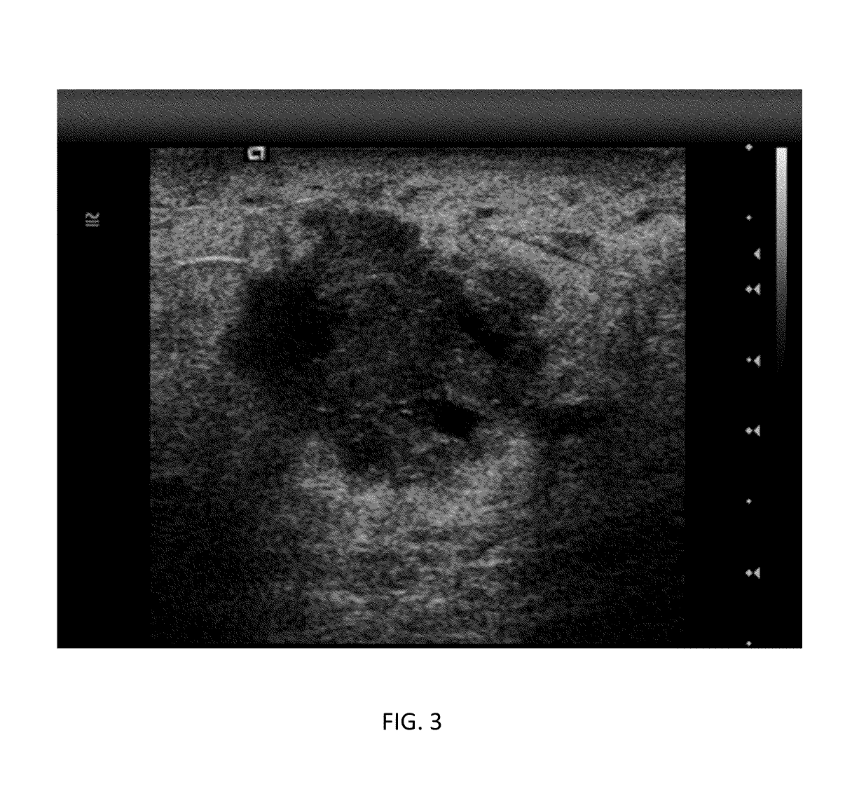

FIG. 3 shows a US image of a breast, where the gray scale of the surrounding region has been standardized.

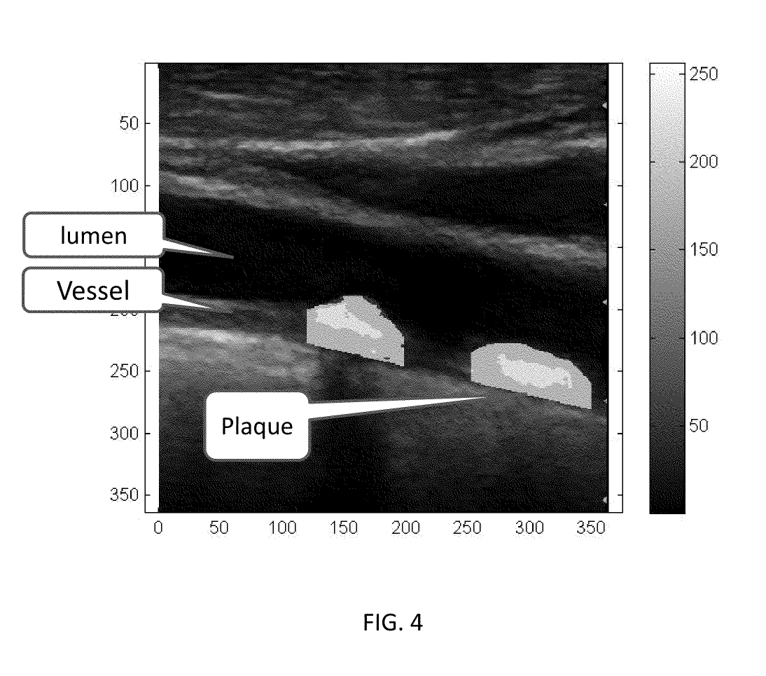

In addition to the feature analysis, the standardized images may be analyzed using the gray scale distribution and higher order pixel characteristics, so as to perform further segmentation of the image. In an example, shown in FIG. 4, two regions of plaque have been segmented based on quantitative analysis of echogenicity. Such segmentation may be done by computer processing means and may be performed either in real time or subsequently. For display purposes, pseudo-color may be used to represent tissue regions, or to show gradations of echogenicity. Since the relative volumes of high- and low-density plaque (plaque heterogeneity) may be of diagnostic value, the segmentation along with a mean value determination for each region may provide sufficient diagnostic information.

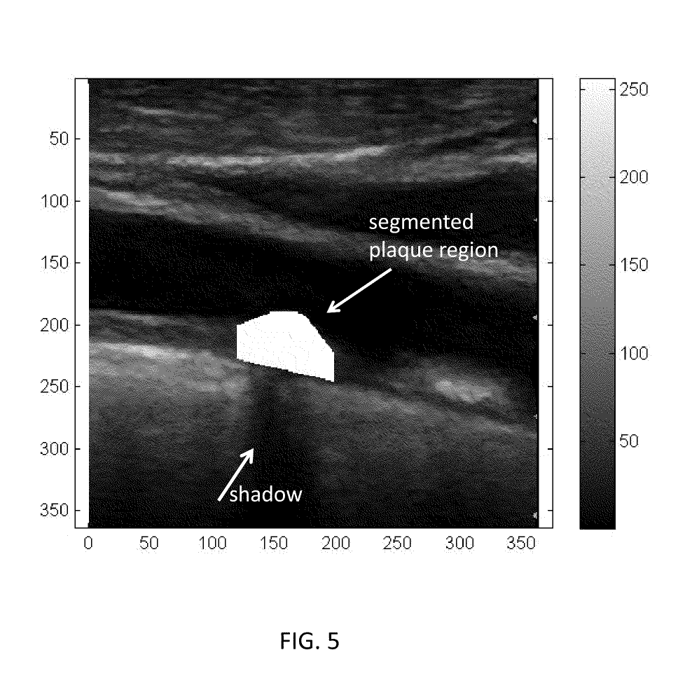

Calcification interferes with ultrasound beam penetration. This results in image shadowing below the calcium region. FIG. 5 is the same image as FIG. 4; however, the plaque is segmented from the vessel without regard to the echogenicity of the plaque so as to clearly show the shadowing (arrow) on the far side far side of the plaque from the sensor. Shadowing may also be detected, for example, by comparing the gray scale value at the same pixel location across images taken at different angles. An adaptive threshold, may be set to identify the shadowing region, which may also be recognizable as having a body tissue speckle characteristic, but with a reduced gray scale value. An additional identifier is that the echo intensity of calcium is bright and will overlay the shadowed region. These characteristics may be alternatively described as relating the extinction along the acoustic ray path to the effective attenuation of the medium (including the effects of scattering).

3D US images of the carotid artery may be acquired by translating an US transducer of the ultrasound imaging device slowly along the neck of a subject for approximately 4 cm. The US probe may be held by a mechanical assembly with a transducer angle rotating around or perpendicular to the skin and to the direction of the scan. Alternatively the transducer may be moved manually. A sequence of two-dimensional images may be saved to a computer workstation, and reconstructed into a 3D image either as they are acquired, or subsequently. The spacing of the 2D images may be determined by the linear or angular speed of motion of the transducer in the direction of scan. An ultrasound contrast agent (UCA) may be used to show the presence of plaque neovasculature. The UCA may be, for example, highly reflective microbubbles which flow along with blood in the vessel and can be destructed by ultrasound waves. The change of plaque intensity before and after the destruction of UCA indicates neovasculature. However, two issues are associated with this technique. One is that FDA requires a warning regarding the safety of UCA. The other is the additional operations required for UCA such as injecting the agent and waiting for its perfusion. An alternative approach of detecting neovasculature is to measure the plaque strain over a cardiac cycle. The strain is caused by the in-fill of neovessels when the arterial pressure changes over the cardiac cycle.

Plaque strain may be detected from the pattern mapping of the coherent RF (radio frequency) acoustic data. Different plaque components have different elasticity, such that their displacement caused by cardiac pressure pulsation is different. As a result, plaque strain may characterize plaque components. Small physical displacements in the data may be detected by cross-correlation processing. A small time window of RF data in one pixel volume (voxel) may be cross-correlated with the RF data in the substantially the same pixel volume of a second image. With a sufficiently high sampling rate with respect to the cardiac cycle, the distance between the correlation peaks at any pixel volume location is a measure of the tissue displacement due to cardiac-induced strain. In addition to the plaque components, this technique may also be used to identify neovasclature. B mode or tissue Doppler data, which is less sensitive to small displacements than RF data, may also be used.

Change of shape and size of a structure caused by hemodynamics, in addition to vessel strain and IMT, may characterize the mechanical properties of a plaque. A method and system are conceived to compute those changes in a cardiac cycle to illustrate or quantify the properties. The change can be the non-overlapped area or the percentage of the total region as a function of some or all of cardiac cycle, blood speed or plaque echogenicity. The change of surface can also be used as a rupture.

A thin fibrous cap may be characteristic of unstable plaque. In US images, the fibrous cap is observed as a bright peripheral region of the plaque between the lumen and the plaque core. The thickness of fibrous cap appears to be significantly different between asymptomatic and symptomatic patients.

An inner boundary separates the fibrous cap from a hypoechoic lipid core and an outer boundary separates the plaque from the surrounding vessel wall and lumen. The fibrous cap thickness may be defined as the distance in the normal direction from the inner boundary to the outer boundary, measured with respect to the vessel axis. The minimal, maximal and average thickness of the fibrous cap may be measured and recorded.

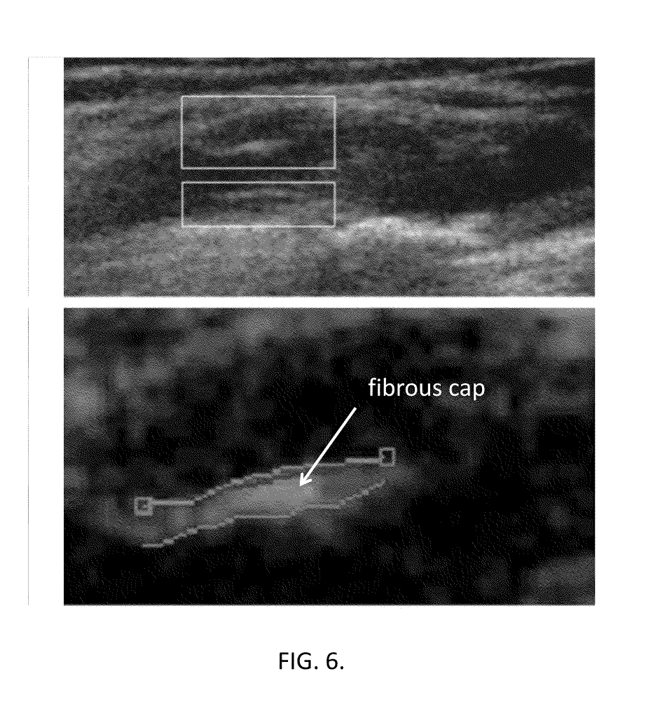

A tracing algorithm similar to that used in intima-media thickness (IMT) determination may be used to determine the thickness of the fibrous cap. US resolution is proportional to sound frequency. A 7.5 MHz operating frequency US imaging device, for instance, has a theoretical resolution of 0.2 mm The IMT algorithm traces the inner and outer boundaries of the fibrous cap by minimizing an energy function. An example of such an analysis is shown in FIG. 6.

Other conventional descriptions of the ultrasound images such as percentage stenosis, and the like, are useful as part of the present method. These descriptions may be determined by observation or by algorithms.

Moreover, although the system and method are being described using the carotid artery as an example other syndromes measurable by ultrasound are similarly characterizable by these techniques and the method may be applied in a variety of diagnostic situations.

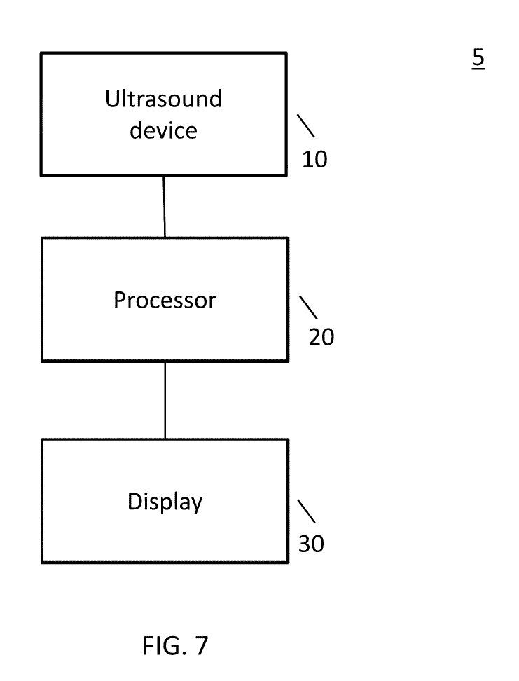

In an aspect, FIG. 7 is a system 5 for performing ultrasound including a ultrasound imaging device 10, an analysis processor 20, which may be a local computer, or be remotely located, and a display 30 which may provide for an operator interface for interacting with the image analysis process, and which may also perform the steps of the methods described below, either fully automatically or guided by the operator. The ultrasound imaging device may be one of a variety of such devices that are currently available, such as a MicroMaxx (SonoSite, Inc., Bothell, Wash.) or iU22 xMATRIX (Philips Healthcare, Andover, Mass.). Such ultrasound imaging devices may include an acoustic signal generator, a transducer capable of transmitting and receiving acoustic energy, and a processor. The processor may be comprised of one or more processing elements and may be segmented into a beamformer, signal processor, image processor, and the like. The specific architecture may be dependent on the design epoch of the device as these functions can be performed by one or more processors, depending on the capability of the electronic components, the throughput requirements, and the like. A display may also be provided for control of the operation and to permit an operator to edit or adjust parameters so as to intervene in an automate analysis where appropriate.

The field of ultrasound imaging continues to develop and new and more capable devices may be introduced in the future. Such ultrasound imaging devices 10 may include, for example sufficient processor resources so as to subsume some or all of the functions of the analysis processor 20 described herein, and may also include an integral display, to perform the function of the display. For example, some or all of the functions of a first processor, which may be the image processor of ultrasound imaging device 10 and a second processor, which may be the analysis processor 20 may be combined in the image processor or other processor of the ultrasound imaging device 10. The allocation of processing functions to the various processing resources, and the packaging of the overall system is a matter of design choice.

The system 5 may also have an interface to a network so as to store or retrieve images and ancillary data. The network may be any of the currently known, or to be developed, techniques for communicating data over a distance, using a local area network (LAN), Internet, and by wired or wireless connections.

The components of the system may be arranged and combined as needed for the configuration of the product, so that the display may be integral or separate from the ultrasound imaging device. The processor in the ultrasound imaging device may perform functions other than the processing of the received acoustic signals to form an ultrasound image. Analysis functions such as the tissue identification, segmentation of the image, and the like, may be performed in the same processor as is being used to produce the image data, another processor within the ultrasound imaging device, or in an analysis processor 20 such as a personal computer (PC) or computer workstation that is in communication with the ultrasound imaging device. The processing of the image data may also be performed by receiving image data that has been stored in an external memory or data base and may be retrieved over a network by the ultrasound system. As such, the network interface may be associated with the ultrasound device 10 or the analysis processor 20, depending on the configuration of the specific system.

A method of identifying tissue types of samples based on analysis of images is described. The method includes the steps of: obtaining images of tissues according to a protocol; extracting features from the images of a specific tissue type using a learning technique; and using the learned features as heuristics to classify portions of images obtained from tissues of an unknown type.

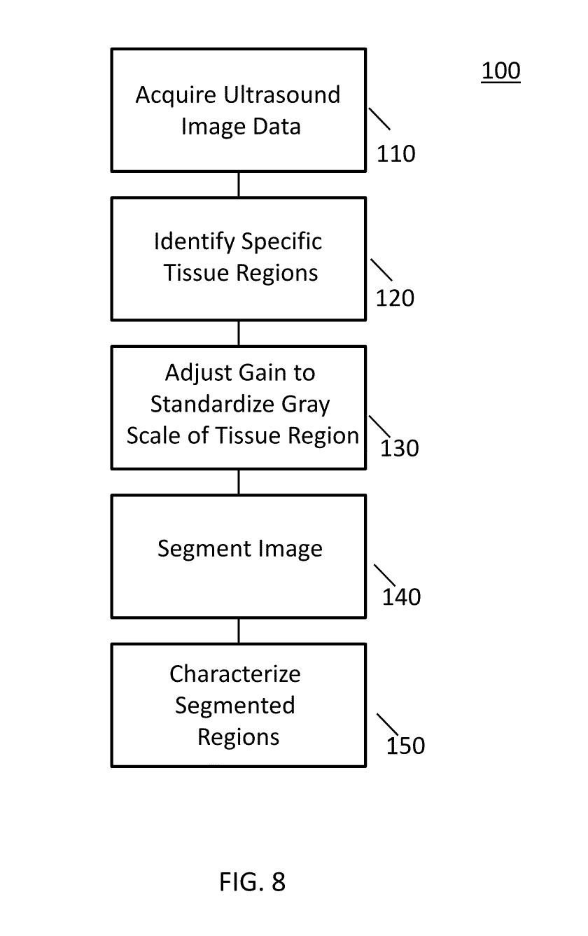

In an example, shown in FIG. 8, the method 100 comprises using an ultrasound device 10 to acquire ultrasound images (step 110) and identifying a specific tissue type in the images (step 120). The gain of the ultrasound device 10 may be adjusted so that the grey scale values associated with the specific tissue type meets a criterion, such as median gray scale value (step 130), so as to standardize the image. The processing to standardize the gray scale and to perform tissue identification, segmentation and tissue analysis may be performed either in the ultrasound imaging device 10, or in an external analysis processor 20. Where the computations associated with standardizing the gray scale are preformed, at least in part, in an analysis processor, the analysis processor 20 may control the sensitivity of the ultrasound imaging device 10 through an interface. The control of sensitivity may include, for example, varying the transmitted power, an acoustic receiver gain, or by adjusting the grey scale in the digital representation of the acoustic data.

The standardized image may be segmented using the identified tissue types so as to differentiate the various regions of interest for analysis (step 140). Selected segmented regions may be further characterized in terms of median gray scale values, higher level features, or the like (step 150).

The step 110 of acquiring images may be performed in real time or the images may be retrieved from a data base such as DICOM (Digital Imaging and Communications in Medicine) where patient history and previously obtained image data may be stored. The standardization step, may be more effective if it is performed in real time, however existing image data may be processed so as to adjust the gray scale to approximate the real-time adjustment. There may be some limits to the dynamic range that may be achieved in historical data processing, however such data may be useful both from the standpoint of diagnosing a particular patient where a temporal history is available in the DICOM database, or for use in training the feature identification algorithms.

The identification of specific tissues (step 120) may be performed initially so as to identify the tissue that is going to be used to standardize the system gain, and may be performed again on the standardized image. That is, step 120 may be performed both before and after performing step 130. The heuristic may be different for each use of step 120.

After identification of the tissue types in the standardized image, the image may be segmented so as to define the boundaries between the tissue types that are characterized by the feature analysis (step 140). A variety of segmentation algorithms have been developed for image processing of the human body, and the selection and use of such algorithms will be familiar to a person of skill in the art. After the regions have been segmented (step 140), each of the tissue regions may be characterized as previously described, so as to make use of the gray scale, texture, and the like.

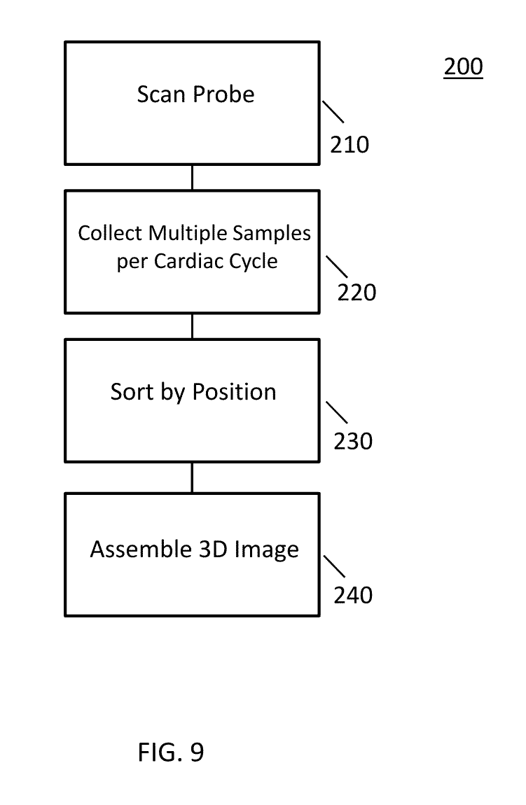

In another aspect, shown in FIG. 9, the method may be used to acquire a three dimensional representation of the region being studied. The sensor head of the ultrasound device 10 may be moved slowly along or across the region to be studied (210). The motion is sufficiently slow such that a plurality of images of substantially the same volume is obtained over one or more cardiac cycles (step 220). The cardiac cycle timing may be obtained by recording EKG (electrocardiogram) data at the time that the images are obtained, or by grouping time spaced images based on correlation or frequency analysis so as to achieve a best match between adjacent spatial images (step 230). After grouping the images so as to comprise images at spatially separated intervals (step 240), for the same place in the cardiac cycle, the images may be further analyzed. The images may be subject to the method 100 so as to segment each of the images, and to fuse the images so as to result in a 3-dimensional segmented rendering of the volume being studied. The results for representative locations in the cardiac cycle may be compared.

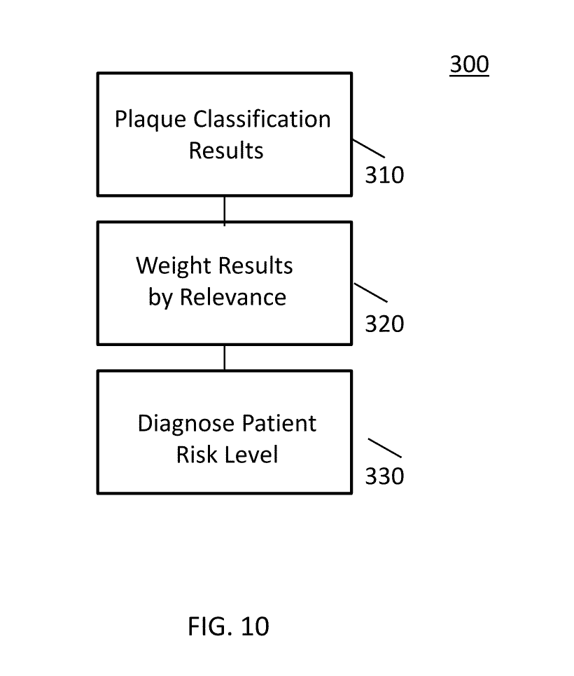

In yet another aspect, shown in FIG. 10, the identified cardiac plaque may be risk scored. The characterized segmented regions obtained in step 150, above, may be analyzed in detail so as to determine specific values of echogenicity, heterogeneity, strain characteristics, fibrous tissue thickness and mechanical properties and calcification. The risk scoring may use a heuristic.

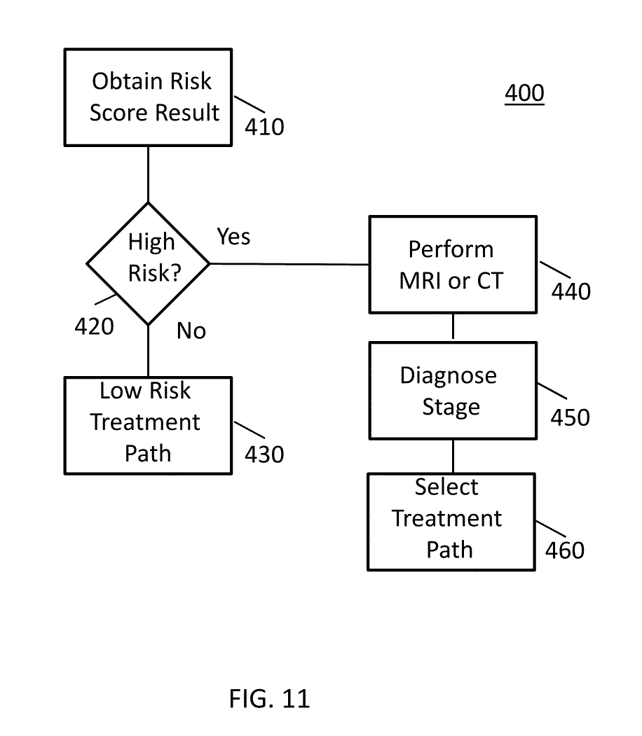

In still another aspect, shown in FIG. 11, a method 300 of diagnosing a patient may use the results of plaque characterization (for example, step 150 or 240) to stage the patient. The quantitative plaque classification results may be applied to a numerical model 320 and the score of the model may classify the plaque as “high-risk” or “low-risk”, or some intermediate classification (step 330). The diagnosis of a patient syndrome is both art and science. So, it may be expected that the model (step 320) is an evolving algorithm, informed by both published research, and by the retrospective analysis of outcomes for patients being evaluated using the system and method described herein.

From a diagnostic viewpoint, the information as to the risk classification of the plaque may be used in a method of determining the treatment for a specific patient (method 400). The risk score result (step 330) may be used, in conjunction with other medical information and patient history to assist medical professionals in determining whether further diagnostic tests are warranted. Such tests are often more expensive and invasive than ultrasound. If the risk score result (step 410) is “low risk” (step 420) the patient may be assigned to a low risk plaque treatment path (step 430). However, if a risk threshold is exceeded, either objectively or on the basis of the combination of the plaque characterization, symptoms, or medical history, the patient may be scheduled for a MRI or CT examination (step 450). The results of step 450, combined with the previously obtained ultrasound plaque assessment may permit the staging of the syndrome (step 460). This classification of the patient may be used to select the appropriate treatment path (step 470).

The images obtained with another imaging modality such as MRI or CT may be selected and registered with the corresponding standardized ultrasound image, and may include the segmentation information of the ultrasound image so as to aid in the diagnostic interpretation of the images obtained from the another imaging modality.

While the methods disclosed herein have been described and shown with reference to particular steps performed in a particular order, it will be understood that these steps may be combined, sub-divided, reordered or repeated to from an equivalent method without departing from the teachings of the present disclosure. Accordingly, unless specifically indicated herein, the order and grouping of steps is not a limitation of the present disclosure or claims

The examples of diseases, syndromes, conditions, and the like, and the types of examination and treatment protocols described herein are by way of example, and are not meant to suggest that the use of the system and method is limited to those named, or the equivalents thereof. As the medical arts are continually advancing, the use of the methods and system described herein may be expected to encompass a broader scope in the diagnosis and treatment of patients. Although only a few exemplary embodiments have been described in detail above, those skilled in the art will readily appreciate that many modifications are possible in the exemplary embodiments without materially departing from the novel teachings and advantages of the techniques disclosed. Accordingly, all such modifications are intended to be included within the scope set forth in the following claims.

What is claimed is:

1. An ultrasound system, the system comprising:
an ultrasound imaging device having a first processor configured to produce image data representing an image of a region of interest of a patient;
a second processor configured to process the image data to obtain a plurality of feature vectors characterizing a sub-region of the image;
wherein the feature vectors are dimensionally reduced and used to identify a specific tissue type based on a heuristic.

2. The system of claim 1, wherein the first processor and the second processor are the same processor.

3. The system of claim 1, wherein a specific tissue type is identified using the heuristic and a sensitivity of the ultrasound device is controlled so that a gray scale distribution value of the image corresponding to the specific tissue type is a predetermined value.

4. The system of claim 3, wherein the predetermined value is a mean gray scale value.

5. The system of claim 3, wherein a plurality of sub- regions of the image data are analyzed so that a tissue type of each sub-region is determined.

6. The system of claim 3, wherein characteristics of a pixel of the image data are determined using a feature vector set of the sub-region surrounding the pixel.

7. The system of claim 3, wherein the identified tissue type is a basis for segmentation of the image.

8. The system of claim 7, wherein a lumen boundary is identified as the boundary between a blood vessel and a region of blood.

9. The system of claim 8, wherein a region of plaque is identified from the segmented data.

10. The system of claim 9, wherein the region of plaque is further segmented based on echogenicity into at least high and low echogenicity regions.

11. The system of claim 6, wherein a temporal series of images is collected for a region of interest.

12. The system of claim 11, wherein a time extent of the temporal series of images is a cardiac cycle.

13. The system of claim 11, wherein the image data of the image is related to a cardiac cycle using EKG data recorded at the same time as the image data.

14. The system of claim 11, wherein the temporal series of images is related to a cardiac cycle by processing images of the temporal series of images so as to identify a periodicity of lumen displacement associated with hemodynamic factors.

15. The system of claim 1, further comprising an interface in communication with a data storage system.

16. The system of claim 15, wherein the data storage system operates in conformance with a Digital Imaging and Communications in Medicine Digital (DICOM) protocol.

17. The system of claim 1, wherein the images are a series of images obtained while moving a sensor head of the ultrasound device with respect to a bodily structure to be examined.

18. The system of claim 2, wherein a plurality of a temporal series of images are processed such that a displacement of voxels from successive images is obtained, and the displacement of voxels is measured over a time period.

19. A method of diagnosing a patient, the method comprising:
receiving image data of a region of interest for a patient, the image data forming an image having a gray scale;
determining a set of feature vectors of the image for a sub-region of the region of interest; and
dimensionally reducing the feature vector set and identifying a tissue type of the sub-region using a heuristic.

20. The method of claim 19, further comprising:
using the identified tissue type to standardize the image gray scale with respect to a predetermined gray scale distribution value for the identified tissue type, by adjusting the gray scale of the image data.

21. The method of claim 19, wherein the step of receiving includes accepting image data from an ultrasound imaging device.

22. The method of claim 19, wherein the step of receiving includes accepting data retrieved from a data base of ultrasound device images.

23. The method of claim 20, further comprising:
determining feature vector sets of regions of the image corresponding to a region of interest and identifying a tissue type for each region based on a heuristic for each tissue type;
segmenting the region of interest based on the identified tissue type of the sub-regions.

24. The method of claim 23, further comprising:
segmenting a region of plaque into at least high and low echolucent regions.

25. The method of claim 24, further comprising:
processing a temporal series of images and determining the stress-strain displacement characteristics of the identified tissue.

26. The method of claim 23, wherein a blood vessel region including a segmented region of plaque is characterized as to at least two of percentage of high and low echolucent material, fibrous cap parameters, degree of stenosis, strain, displacement, plaque surface smoothness, or extent of calcification.

27. The method of claim 26, wherein the characterized plaque is used to compute a risk score the patient in accordance with a risk score heuristic.

28. The method of claim 27, further comprising: using the risk score is used determine whether a further diagnostic test is performed.

29. The method of claim 28, wherein the further diagnostic test is obtaining a magnetic resonance imaging (MRI) image of the region of interest.

30. The method of claim 19, further comprising: determining the heuristic using supervised training.

31. The method of claim 19, further comprising: determining the heuristic using unsupervised training.

32. The method of claim 23, further comprising: registering the segmented image with respect to an image obtained using another imaging modality.

33. The method of claim 32, wherein the image obtained using another imaging modality is a magnetic resonance imaging (MRI) image.

34. A computer program product, stored in a non-transient computer-readable medium, comprising:
instructions interpretable by a processor to cause the processor to:
receive image data image of a region of interest for a patient;
determine a set of feature vectors for a sub-region of the region of interest; and
dimensionally reduce the feature vector set and identify a tissue type of the sub-region using a heuristic.

35. The computer program product of claim 34, wherein when the identified tissue type is suitable for image standardization:
standardize the gray scale of the image with respect to a predetermined gray scale distribution value for the identified tissue type by adjusting the grey scale of the image.

36. The computer program product of claim 35, wherein the standardized image is segmented based on a plurality of identified tissue types.

37. The computer program product of claim 35, wherein the tissue types are identified on a pixel basis, using a feature vector set of a surrounding sub-region.

37. The computer program product of claim 35, wherein the standardized image is registered with respect to an image of the patient obtained using another imaging modality.

38. The computer program product of claim 36, further comprising:
segmenting a region of plaque into at least high and low echolucent regions.

39. The computer program product of claim 38, wherein a blood vessel region including a segmented region of plaque is characterized as to at least two of percentage of high and low echolucent material, fibrous cap parameters, degree of stenosis, strain, displacement, plaque surface smoothness, or extent of calcification.

40. The computer program product of claim 39, wherein the characterized blood vessel region is used to compute a risk score the patient in accordance with a risk score heuristic.

41. The computer program product of claim 35, wherein the grey scale of the image is standardized by controlling a parameter of an ultrasonic imaging device when the image is being obtained.

42. The computer program product of claim 41, wherein the parameter is a gain setting.
